(19) United States
(12) Patent Application Publication
(10) Pub. No.: US 2018/0092538 A1
(43) Pub. Date: Apr. 5, 2018

(54) SYSTEMS AND METHODS FOR DUAL-MODE IMAGING USING OPTICAL COHERENCE TOMOGRAPHY AND FLUORESCENCE IMAGING

(71) Applicants: Nandini Rajan, Lincoln, MA (US); Andrew M. Siegel, Arlington, MA (US); Angela M. Belcher, Cambridge, MA (US); Neelkanth M. Bardhan, Cambridge, MA (US)

(72) Inventors: Nandini Rajan, Lincoln, MA (US); Andrew M. Siegel, Arlington, MA (US); Angela M. Belcher, Cambridge, MA (US); Neelkanth M. Bardhan, Cambridge, MA (US)

(73) Assignee: Massachusetts Institute of Technology, Cambridge, MA (US)

(21) Appl. No.: 15/411,704

(22) Filed: Jan. 20, 2017

Related U.S. Application Data
(60) Provisional application No. 62/347,477, filed on Jun. 8, 2016.

(51) Int. Cl. G01B 9/02; A61B 5/00

(52) U.S. Cl. CPC A61B 5/0066; G01B 9/02091; A61B 5/0073

(57) ABSTRACT

Systems and methods are provided to obtain optical coherence tomography data and fluorescence imaging data from an object. The systems and methods use a single light source to illuminate and excite fluorescence in the object and a common optical path including an optical fiber to provide illumination and excitation light and collect reflected and emitted light at the object. The optical coherence tomography data and fluorescence imaging data can be combined to produce morphological images of an object with additional location-specific fluorescence cues.

### RELATED APPLICATION

This patent application claims priority to U.S. Provisional Patent Application No. 62/347,477, filed Jun. 8, 2016, the entire contents of which is incorporated herein by reference.

### STATEMENT OF GOVERNMENT INTEREST

This invention was made with Government support under Grant No. U54 CA151884 awarded by the National Institutes of Health and under Contract No. FA8721-05-C-0002 awarded by the U.S. Air Force. The Government has certain rights in the invention.

### BACKGROUND

Optical coherence tomography (OCT) is an optical imaging technique that can generate high-fidelity, micron-scale, three-dimensional imagery of objects such as biological tissues. Some OCT systems can acquire image data transcutaneously (i.e., through the skin). Some OCT systems can include an endoscopic probe to acquire image data through a lumen of the body (e.g., large blood vessels or the esophagus). Reconstructed OCT images provide three-dimensional morphological information that enables clinicians to examine biological tissue in detail at depths of up to several millimeters below the tissue surface.

Targeted fluorescent contrast agents are a type of functionalized probe that bind to specific receptors located in or on a tissue. For example, the receptors may be specific to a type of cancerous tumor. Because the fluorescent contrast agents are conjugated directly to their targets, the resulting fluorescent image data created upon excitation of the contrast agent provides the ability to discriminate between tissues that do and do not manifest the specific receptor.

Conventional bimodal imaging systems that include both OCT and fluorescence imaging capabilities include two or more light sources to provide (e.g., a dedicated light source for OCT imaging and a separate dedicated light source to excite fluorescence in the object.)

### SUMMARY

Taught herein are systems and methods for dual-mode imaging of an object with both optical coherence tomography techniques and fluorescence imaging techniques using a single light source. In some embodiments, the single light source provides illumination and excitation light to synchronously obtain both OCT image data and fluorescence image data of the object.

In some embodiments taught herein, an imaging system is disclosed for generating fluorescence image data and optical coherence tomography data of an object. The imaging system includes a light source configured to provide light for illumination of at least a portion of the object and for excitation of fluorescence in at least the portion of the object. The imaging system also includes an optical coherence tomography subsystem configured to provide optical coherence tomography data of the portion of the object and configured to use light from the light source. The imaging system also includes a fluorescence measurement subsystem configured to provide fluorescence image data of the portion of the object and configured to use light from the light source.

In some embodiments taught herein, a method of imaging an object is disclosed. The method includes providing light from a light source for illumination of at least a portion of the object and an optical coherence tomography reference arm and for excitation of fluorescence in at least a portion of the object. The method also includes directing the light for illumination of the portion of the object and the light for excitation of fluorescence onto the portion of the object using an optical fiber. The method also includes collecting, using the optical fiber, reflected light from the portion of the object and fluorescence emitted at one or more emission wavelengths in the portion of the object. The method also includes detecting the collected reflected light from the portion of the object and reference light from the reference arm to provide optical coherence tomography data representative of the portion of the object. The method also includes detecting the fluorescence emitted from the portion of the object to provide fluorescence image data for the portion of the object.

### BRIEF DESCRIPTION OF THE DRAWINGS

The skilled artisan will understand that the drawings are primarily for illustrative purposes and are not intended to limit the scope of the subject matter described herein. The drawings are not necessarily to scale; in some instances, various aspects of the subject matter disclosed herein may be shown exaggerated or enlarged in the drawings to facilitate an understanding of different features. In the drawings, like reference characters generally refer to like features (e.g., functionally similar or structurally similar elements).

The foregoing and other features and advantages provided by the present disclosure will be more fully understood from the following description of exemplary embodiments when read together with the accompanying drawings, in which:

FIG. 1A schematically depicts a block diagram illustrating various portions of an imaging system in accordance with some embodiments of the present disclosure.

FIG. 1B schematically depicts a block diagram illustrating various portions of an imaging system in accordance with some embodiments of the present disclosure.

FIG. 2A schematically depicts a block diagram illustrating an optical coherence tomography subsystem of an imaging system using a single light source and multiple detectors in accordance with some embodiments of the present disclosure.

FIG. 2B schematically depicts a block diagram illustrating a fluorescence imaging subsystem of the imaging system using the single light source and multiple detectors in accordance with some embodiments of the present disclosure.

FIG. 2C schematically depicts the imaging system using the single light source and multiple detectors for synchronously generating fluorescence image data and optical coherence tomography data of an object according to some embodiments of the present disclosure.

FIG. 3A schematically depicts a block diagram illustrating the optical coherence tomography subsystem of an imaging system using the single light source and a single detector in accordance with some embodiments of the present disclosure.

FIG. 3B schematically depicts a block diagram illustrating the fluorescence imaging subsystem of the imaging system using the single light source and the single detector in accordance with some embodiments of the present disclosure.

FIG. 3C schematically depicts the imaging system using the single light source and the single detector for synchronously generating fluorescence image data and optical coherence tomography data of an object according to some embodiments of the present disclosure.

FIG. 4 depicts an excitation-emission plot for various formulations of contrast agents in accordance with some embodiments of the present disclosure.

FIG. 5 is a flow diagram of a method of imaging an object in accordance with some embodiments of the present disclosure.

FIG. 6 schematically depicts a tissue being imaged by an imaging system according to some embodiments of the present disclosure.

FIG. 7 schematically depicts an endoscope of the imaging system in accordance with some embodiments of the present disclosure.

FIG. 8 schematically depicts a computing device suitable for use with some embodiments of the present disclosure.

Additional features, functions and benefits of the disclosed methods, systems and media will be apparent from the description which follows, particularly when read in conjunction with the appended figures.

### DETAILED DESCRIPTION

Systems and methods taught herein employ optical coherence tomography and fluorescence imaging to create morphological images of an object with additional location-specific fluorescence cues. The resulting dual-mode OCT and fluorescence images can be sampled at a spatial and temporal resolution commensurate with scientific or diagnostic need. The systems and methods use a single light source to synchronously provide optical coherence tomography data and to excite fluorescence in the object. The use of a single light source for this dual purpose simplifies the optical path, enables faster data acquisition, and reduces system cost. In some embodiments, the systems and methods employ a common optical path (e.g., a single optical fiber) for providing excitation and illumination light to the object and for collecting reflected and fluorescence light from the object. The use of a common optical path immediately prior to and from the object (e.g., the single optical fiber) enables simultaneous temporally and spatially synchronized collection of elastically scattered (reflected) light and inelastically scattered (fluorescent) light from the object. Use of the common optical path immediately prior to and from the object (e.g., the single optical fiber) also creates spatial co-registration of reflected light image data and fluorescence image data from the object.

As used herein, the term “fluorescence” includes all modes of photo-generation and photoemission such as fluorescence, phosphorescence, plasmon resonance scattering, and other forms of luminescence.

As used herein, the term “emit” refers to light production through any known method including emission, elastic or inelastic scattering, or resonance.

As used herein, the term “fluorescence contrast agent” can refer to both extrinsic fluorescent species (e.g., biochemical labels or markers bound to particular components, structures, or tissue types) and to endogenous fluorescent species whether native or arising from, for example, genetic manipulation (e.g., autofluorescence from tissue components).

As used herein, the term “simultaneous” refers to multiple measurements occurring at the same time but not necessarily at the same object location.

As used herein, the term “synchronous” refers to two or more measurements occurring both at the same time (simultaneously), and with a static relationship in regards to time or space. More specifically, “synchronous OCT and NIRF imaging” refers to imagery collected simultaneously by both modalities as they measure the same region of an object during the same period of time (i.e. same time and same space).

Example methodologies and systems are described herein to facilitate synchronous acquisition of optical coherence tomography (OCT) image data and fluorescence image data for biological structures such as tissues. Some embodiments involve the use of a single light source to provide the OCT imaging light and fluorescence excitation light to excite a fluorescence contrast agent in or on the object. In some embodiments, the illumination light and the returning light travel on a common optical path immediately prior to and after the object through an optical fiber. The use of a single light source to illuminate the object for both OCT and fluorescence imaging can improve imaging throughput, simplify optical setups, enable further miniaturization of the probe apparatus, and reduce system cost.

OCT is a powerful imaging technique that can generate high-fidelity, micron-scale, three-dimensional imagery of objects such as biological tissues. Clinicians can use OCT to examine tissue in great detail. OCT image analysis has historically been performed by trained human eyes or computer-aided techniques to identify morphological anomalies to indicate the presence of diseased tissue. Fluorescence imaging can be used to characterize tissue with high-fidelity but low spatial resolution. In general, the lateral spatial resolution of the fluorescence imaging signal will be lower than that of the OCT signal due to the optical scattering properties of the tissue. In addition, the fluorescence measurement provides no axial (depth) resolution because the fluorescence imaging measurement is spectrally incoherent with respect to the pump source. A dual-mode approach employing both OCT and fluorescence imaging has the potential to create images with high-resolution spatial morphology superimposed with automated identification of tissues of interest such as tumors.

Current systems for dual-mode imaging using OCT and fluorescence imaging employ multiple light sources operating sequentially to supply light for OCT imaging and light for fluorescence imaging. Systems with multiple light sources are generally more complex and are more difficult to build and maintain than systems of the present disclosure that use a single light source. Because light sources are often the most expensive part of the system, the need for multiple light sources increases total system cost. Sequential operation of multiple light sources as performed in current systems necessitates longer acquisition times for both OCT and fluorescence data as the acquisition cannot be obtained synchronously. Some current systems for dual-mode imaging using OCT and fluorescence imaging employ multiple optical fibers to deliver light to the object and collect light returning from the object. As a result, these systems have a probe head size immediately prior to the object that prevents the probe from being inserted into small lumens of the body such as small blood vessels or fallopian tubes. Some embodiments of the present disclosure have a probe head size that can include an endoscope and can be used to image within small lumens such as small blood vessels or fallopian tubes. Some embodiments of the present disclosure employ a small probe head size and use a single light source to perform both OCT and fluorescence imaging in real-time synchronously.

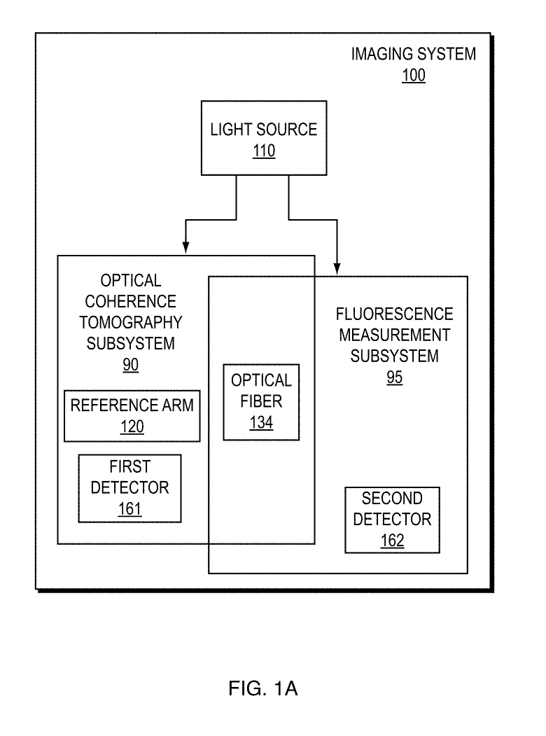

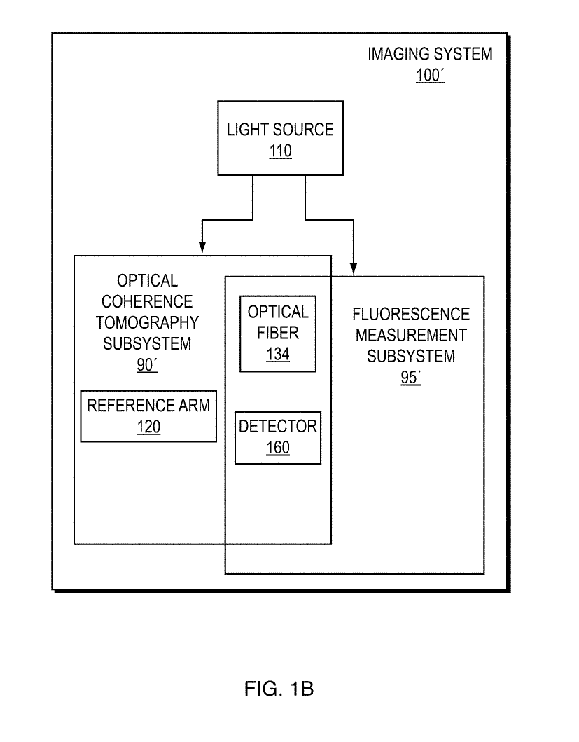

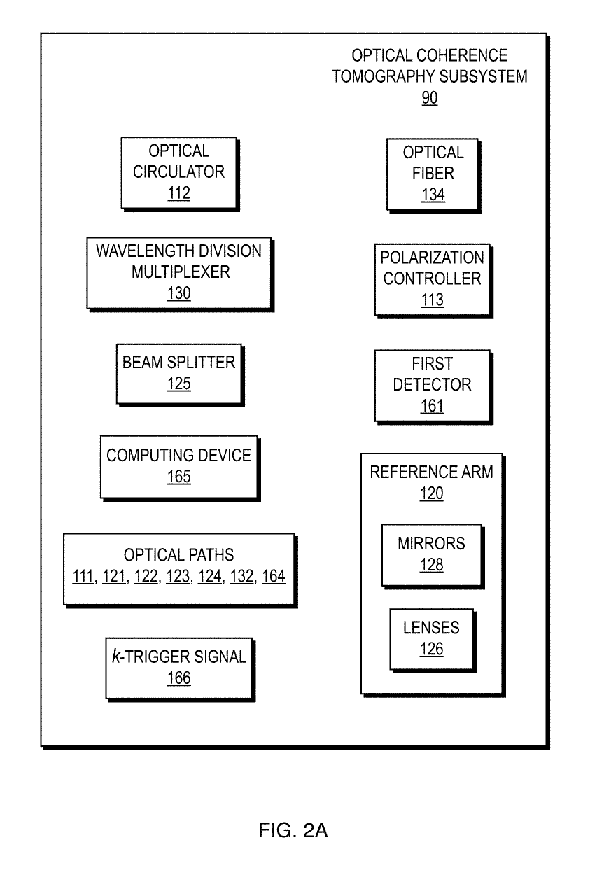

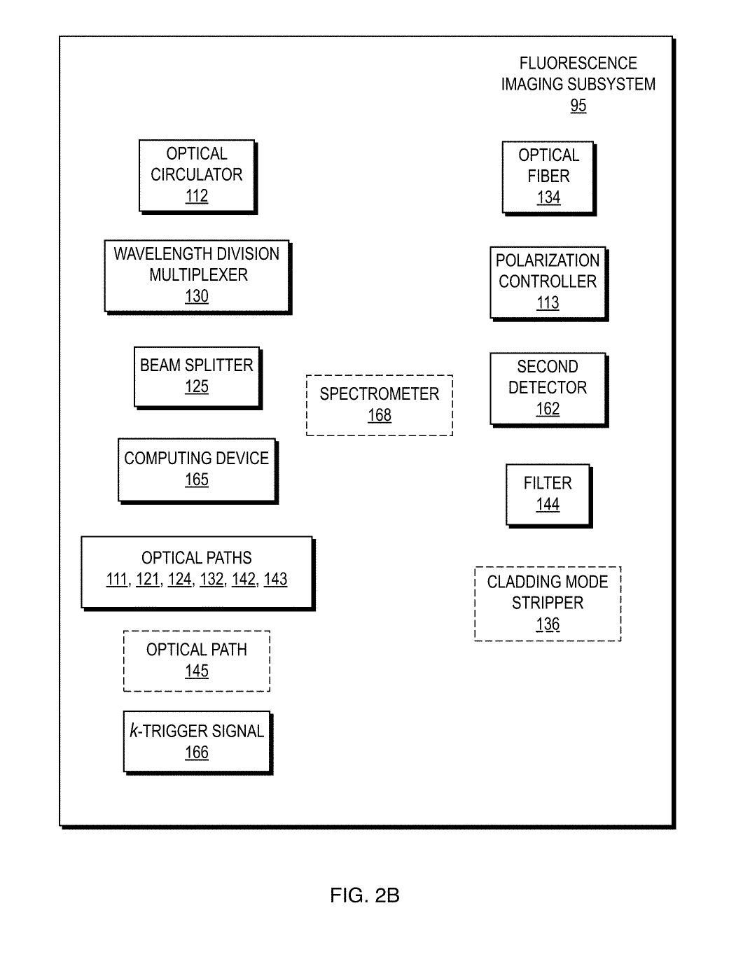

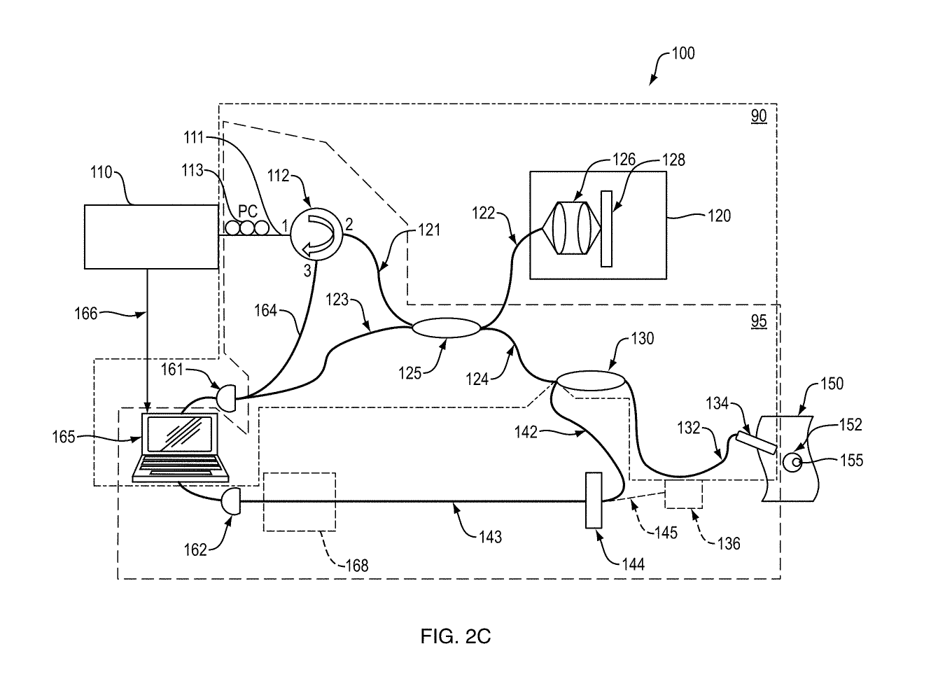

FIG. 1A depicts a block diagram illustrating components of an exemplary imaging system 100 for generating fluorescence image data and OCT data of an object using a single light source 110 in accordance with some embodiments. The system 100 includes an optical coherence tomography subsystem 90 or first imaging subsystem to image an object and provide OCT data and a fluorescence measurement subsystem 95 or second imaging subsystem to image the object and provide fluorescence image data. The optical coherence tomography subsystem 90 and the fluorescence measurement subsystem 95 are both configured to use the light from the light source 150. In some embodiments, use of a single light source 110 to generate both OCT data and fluorescence image data can simplify the optical setup, improve system reliability, reduce the number of optical components in the system, and reduce the total cost of the dual-mode system. Imaging system 100 is described in greater detail below with reference to FIGS. 2A-2C.

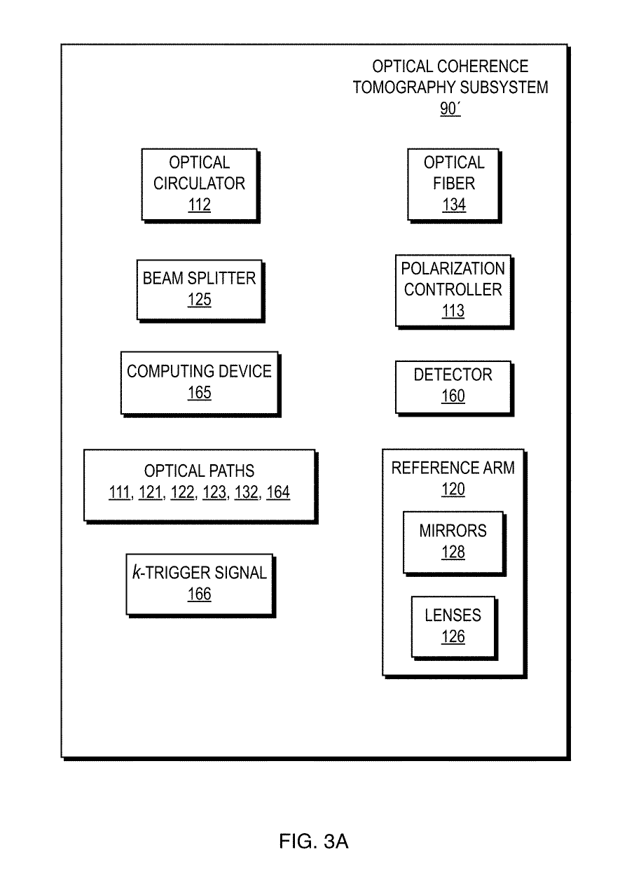

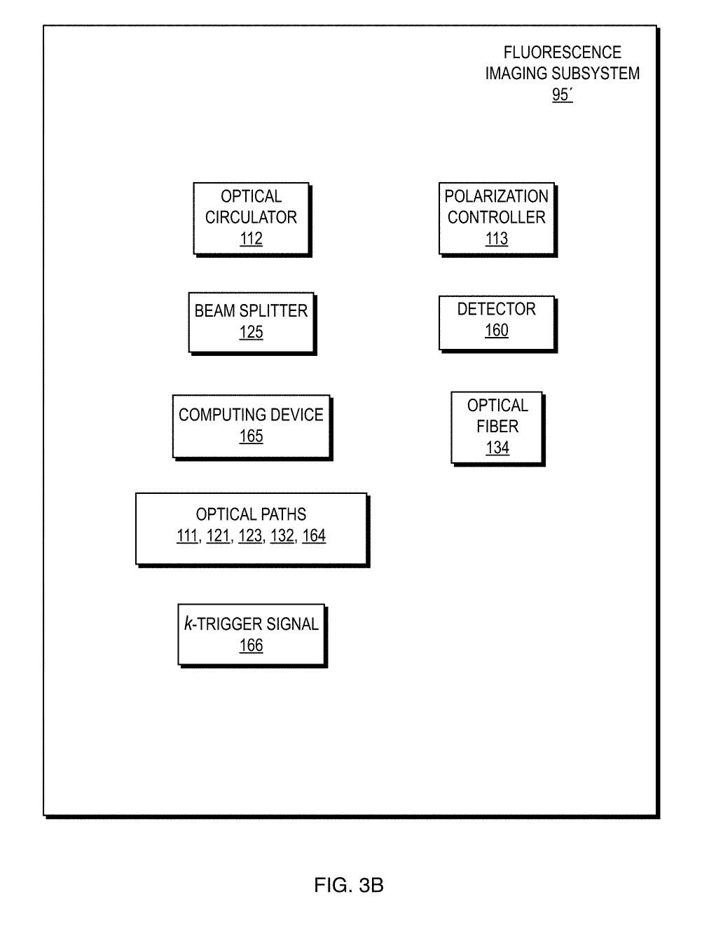

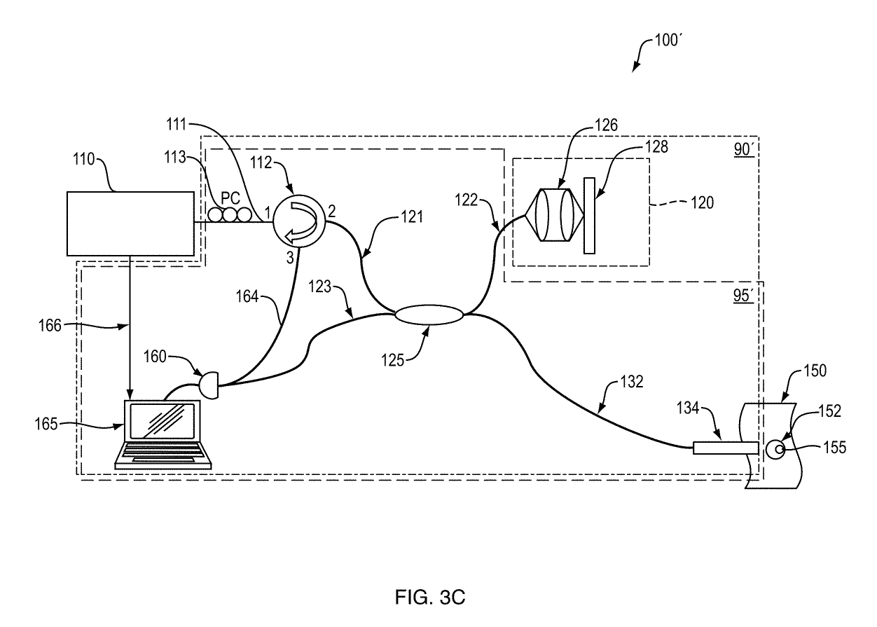

FIG. 1B depicts a block diagram illustrating components of an exemplary imaging system 100′ for generating fluorescence image data and OCT data of an object using the single light source 110 in accordance with some embodiments. The system 100′ includes an optical coherence tomography subsystem 90′ or first imaging subsystem to image an object and provide OCT data and a fluorescence measurement subsystem 95′ or second imaging subsystem to image the object and provide fluorescence image data. The optical coherence tomography subsystem 90′ and the fluorescence measurement subsystem 95′ are both configured to use the light from the light source 150. Imaging system 100′ is described in greater detail below with reference to FIGS. 3A-3C.

Certain elements of the system 100 belong exclusively to one subsystem, e.g., the reference arm 120 belongs exclusively to the optical coherence tomography subsystem 90, 90′. However, some elements of the system 100, 100′ such as an optical fiber 134 that provides and collects light at the object can be considered in some embodiments as part of the fluorescence measurement subsystem 95, 95′, as part of the optical coherence tomography subsystem 90, 90′, or shared by both subsystems. Common use of the optical fiber 134 for both subsystems to provide and collect light at the object can provide advantages such as a reduction in size of a probe of the system at the object, fewer optical components in the probe, and co-registration of OCT data and fluorescence image data. As another example, the optical coherence tomography subsystem 90 and the fluorescence measurement subsystem 95 can share the same detector 160 in some embodiments. In other embodiments, the optical coherence tomography subsystem 90′ uses a first detector 161 while the fluorescence measurement subsystem 95′ uses a second detector 162.

Some embodiments of the system 100 are described in greater detail with reference to FIGS. 2A-2C. The system 100 can include the optical coherence tomography subsystem 90 and the fluorescence imaging subsystem 95 in some embodiments. The dual-mode imaging system 100 uses the light source 110 to illuminate a portion 152 of an object 150 and synchronously produces OCT data and fluorescence image data of the portion 152 of the object 150. In some embodiments, the system 100 can combine the OCT data and fluorescence image data into a combined image containing OCT and fluorescence information. In some embodiments, the system 100 uses the OCT data and fluorescence image data to generate high-resolution axial and morphological images of the object 150 combined with specific labeling by fluorescence in the object 150.

In some embodiments, the optical coherence tomography subsystem 90 can include an optical circulator 112, a beam splitter 125, a computing device 165, an optical fiber 134, a k-trigger signal 166, a first detector 161, a wavelength division multiplexer 130, a polarization controller 113, optical paths 111, 121, 122, 123, 124, 132, 164, and a reference arm 120 including mirrors 128 and lenses 126 as shown in FIG. 2A.

In some embodiments, the fluorescence imaging subsystem 95 can include the optical circulator 112, the wavelength division multiplexer 130, optical paths 111, 121, 124, 132, 142, 143, a second detector 162, a polarization controller 113, a filter 144, a k-trigger signal 166, the optical fiber 134, the beam splitter 125, and the computing device 165 as shown in FIG. 2B. The fluorescence imaging subsystem 95 can include a cladding mode stripper 136, an optical path 145, and a spectrometer 168.

A schematic of the dual-mode imaging system is shown in FIG. 2C according to some embodiments of the present disclosure. The light source 110 is configured to provide light at one or more wavelengths for illumination of the portion 152 of the object 150 and excitation of fluorescence in the portion 152 of the object 150. In some embodiments, the light source 110 can be a swept source, i.e., the output wavelength of the light source 110 can be controllably varied. For example, the light source 110 can output light in a wavelength range that lies within the range of 750 nm to 850 nm in some embodiments. In some embodiments, the light source 110 outputs light at a center wavelength of 800 nm with a bandwidth of about 10 nm. In some embodiments, the light source 110 can output light in a wavelength range that lies within the range of 1200 nm to 1500 nm. In some embodiments, the light source 110 outputs light at the center wavelength of 1310 nm with a bandwidth of about 110 nm. In accordance with some embodiments, the swept light source 110 can be used in combination with components of the optical coherence tomography subsystem 90 to perform frequency- or Fourier-domain OCT techniques such as time-encoded frequency-domain OCT. In frequency-domain OCT techniques, image contrast can be obtained without the need to change the path length of the reference arm. In some embodiments, the wavelength of the light source 110 can be swept on and off of a resonance peak of the fluorescence contrast agent 155. In some embodiments, the light source 110 can have a gap in the range of swept wavelengths to prevent excitation of a fluorescence contrast agent 155 on some passes of the sweep to maximize the productive life-span of the fluorescence contrast agent 155, which can be affected by photobleaching.

In accordance with various embodiments, the wavelength or wavelength range of the light produced by the light source 110 can penetrate deeply enough into the object 150 to provide OCT data. In some embodiments, the wavelength or wavelength range of the light produced by the light source 110 can be on resonance to excite a fluorescence contrast agent 155 in the object 150.

In some embodiments, the system 100 is configured to both direct light from the light source 110 onto the portion 152 of the object 150 and collect reflected and emitted light at one or more emission wavelengths from the object 150 on a common optical path 132 through the optical fiber 134. By providing and receiving light at the object 150 through the single optical fiber 134, the size of an associated probe instrument can be reduced over a probe instrument with multiple optical fibers and associated optics. Such minimization of the size of the probe instrument can enable the system 100 to take measurements in small or constricted lumens such as small blood vessels or fallopian tubes. In some embodiments, the optical fiber 134 can be partially or wholly incorporated into an endoscope 170 to protect the probe instrument or system 100 from environmental factors such as moisture or heat.

Use of the common optical fiber 134 can provide co-registration or self-registration of the collected reflected light and the collected emitted light at the first detector 161 and the second detector 162. In particular, passage of the collected reflected light and collected emitted light pass through the same distal optics 138 (if present) and the same optical fiber 134 at the object 150 causes the magnification and position of the field of view for each of the two modes to be identical. Co-registration of the collected reflected light and the collected emitted light can improve computational speed during image reconstruction by the computing device 165 because additional processing steps including landmark identification or image shifting can be avoided or minimized.

In some embodiments, the optical fiber 134 includes a double-clad fiber. For these embodiments, the imaging system 100 can be configured such that the collected light reflected from the object 150 travels in a single-mode inner core of the double-clad fiber and the fluorescence light travels in a multi-mode outer core of the double-clad fiber. In some embodiments, the optical fiber 134 includes a single-mode optical fiber. In these embodiments, the imaging system 100 can be configured such that the collected light reflected from the object travels in a single-mode inner core of the single-mode optical fiber while the fluorescence light travels in a cladding of the single-mode fiber. A cladding-mode stripper 136 or other optical extraction device can be included in the system 100 in certain embodiments to couple the fluorescence out of the cladding of the single-mode fiber.

In accordance with some embodiments, the optical fiber 134 can be configured to collect reflected light having a wavelength in the range of 750 nm to 850 nm. In accordance with various embodiments, the optical fiber 134 can be configured to collect reflected light having a wavelength in the range of 650 nm to 1800 nm and to collect fluorescence having a wavelength in the range of 650 nm to 1800 nm. In some embodiments, the optical fiber 134 can be configured to collect reflected light having a wavelength in the range of 750 to 950 nm and to collect fluorescence having a wavelength in the range of 1100 nm to 1800 nm.

In some conventional systems that require multiple light sources to generate OCT data and fluorescence image data, each of the multiple light sources is sequentially directed at the object. In contrast, the light source 110 of the present disclosure can be used to synchronously illuminate and excite fluorescence in the object 150. Because of this, the OCT data and the fluorescence image data can also be detected synchronously at the first detector 161 and the second detector 162 or at the same detector 160 in some embodiments of the present disclosure. In some embodiments, when the data is collected synchronously, data acquisition (and, in particular, OCT data acquisition) can occur at full speed because temporal switching between light sources is unnecessary.

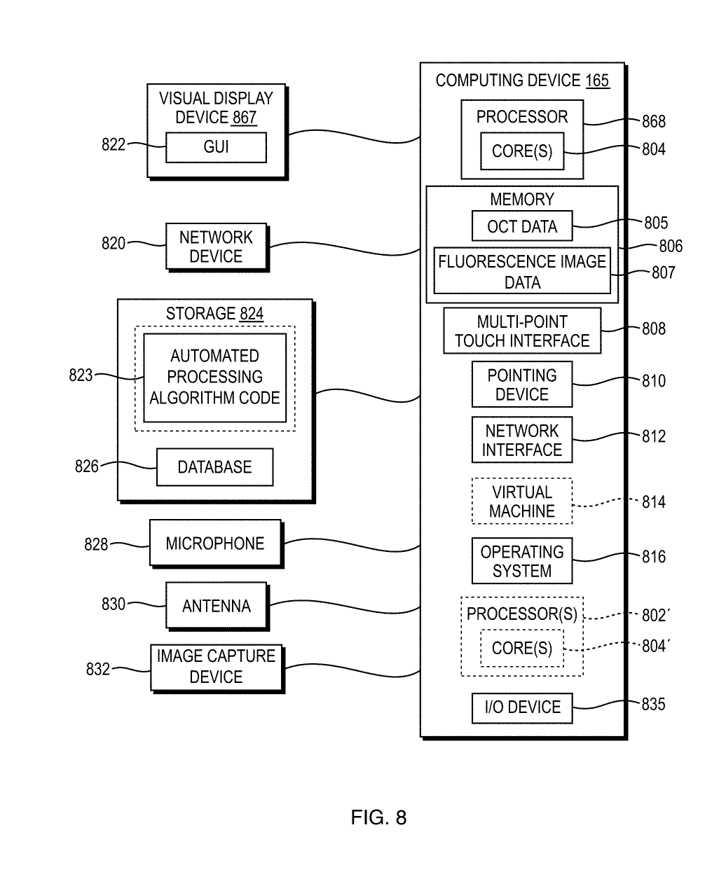

In some embodiments, the OCT data and the fluorescence image data can be combined by the computing device 165 into one or more spatially registered dual-mode images that include high-resolution views of morphological features of the object 150 and that highlight localized features of the object 150 corresponding to locations of fluorescence. In accordance with some embodiments, the computing device 165 can receive a k-trigger signal 166 from the light source 110 to synchronize the frequency sweep of the light source 110 to the signals acquired from the first detector 161. An exemplary computing device 165 is described in greater detail below with reference to FIG. 8.

In some embodiments, the optical coherence tomography subsystem 90 includes one or more of an optical circulator 112, a beam splitter 125, the reference arm 120, the first detector 161, a wavelength-division multiplexer (WDM) 130 and the computing device 165. Various components of the optical coherence tomography subsystem 90 can be connected by optical paths. Optical paths can be delineated in free space or can include one or more optical fibers. Components of the optical coherence tomography subsystem 90 can be discrete optical components mounted separately or mounted integrally. In some embodiments, the components of the optical coherence tomography subsystem 90 can be compatible with optical fibers. In some embodiments, the fluorescence measurement subsystem 95 includes components that are shared with the optical coherence tomography subsystem 90 such as the beam splitter 125, the optical circulator 112, the WDM 130, and the computing device 165. In some embodiments, the fluorescence measurement subsystem includes one or more filters and detectors 162. In some embodiments, the fluorescence measurement subsystem can also include a spectrometer 168. In some embodiments, the system 100 includes a fluorescence contrast agent 155 that emits fluorescence at one or more emission wavelengths and which can be targeted to a specific binding target on the object 150. The fluorescence contrast agent 155 will be described in greater detail below.

In use, light from the light source 110 travels on a path 111 to the optical circulator 112. In some embodiments, a polarization controller 113 is placed along the path 111 to allow adjustment of the polarization of the light from the light source 110. The polarization controller 113 can include manual adjustment or automated adjustment. The optical circulator 112 can act to optically isolate the light source 110 from the remainder of the system 100 to prevent light returning from the system from destabilizing the source. The light then passes along a path 121 to the beam splitter 125. In some embodiments, the beam splitter 125 is a 50/50 beam splitter. In some embodiments, the beam splitter 125 divides the light into unequal portions, e.g., a 75/25 beam splitter or a 90/10 beam splitter. A portion of the light exiting the beam splitter 125 follows a path 122 to the reference arm 120 while the remaining portion of the light follows a path 124 to the WDM 130. Light exiting the WDM 130 follows a path 132 to the optical fiber 134 to illuminate and excite fluorescence in the portion 152 of the object 150.

Reflected collected light and emitted fluorescence from the object 150 return through the optical fiber 134 and along path 132 to the WDM 130. The WDM 130 separates light collected from the object 150 into different paths based on wavelength. For example, the WDM 130 can send collected reflected light at a first wavelength back along the path 124 to the beam splitter 125 and can send fluorescence at a second wavelength onto a path 142 to a filter 144. In some embodiments, the filter 144 can block wavelengths of light corresponding to the illumination light from the light source 110. In some embodiments, the detector 162 has preferential sensitivity to the wavelengths corresponding to the fluorescence light. In such embodiments, the filter 144 may not be required. Although the WDM 130 is shown with two outputs in FIG. 2C, it will be apparent to one skilled in the art that any number of outputs may be used to meet application-specific requirements. In embodiments that employ two or more distinct fluorescence contrast agents 155, the fluorescence light from each type of fluorescence contrast agent 155 may be separated and measured using any of a variety of different methods. For example, in some embodiments, the WDM 130 may perform spectral selection and direct different spectral portions of the fluorescence light to different output paths. The different output paths can then each connect to a detector and can include a bandpass filter centered on one fluorescence wavelength. As another example, in some embodiments, the total fluorescence light can exit the WDM 130 initially on the optical path 142 and can then be split into separate paths. Each path can connect to a detector and can include a bandpass filter centered on one fluorescence wavelength. As another example, the computing device 165 can record and average signals received at the detector 162 sequentially in time as a form of time-division multiplexing or temporal filtering. The time-dependent signals can represent the distinct swept-source excitation wavelength ranges corresponding to the excitation bands for each of the fluorescence contrast agents and can represent each of the multiple fluorescence contrast agents in turn.

Reference light returns from the reference arm 120 along path 122 to the beam splitter 125. Upon reaching the beam splitter 125, the reference light and the collected reflected light can be split by the beam splitter 125 directing a portion along onto a path 123 to the first detector 161 and the remaining portion along the path 121 back to the optical circulator 112. The optical circulator 112 passes the portion of reference light and the collected reflected light received from path 121 along a path 164 to the first detector 161. In some embodiments, the difference between the sum of the lengths of optical paths 164 and 121 and the length of optical path 123 is small compared to a coherence length of the light source 110. The reference light and collected reflected light combine interferometrically at the first detector 161 to form an OCT signal detected by the first detector 161. Output from the first detector 161 can be received by the computing device 165 to produce OCT data.

In some embodiments, the first detector 161 is a balanced photodetector such as a balanced photo-diode. In some embodiments, the balanced photodetector can operate in a difference mode wherein the output signal is representative of OCT data.

In some embodiments, the reference arm 120 can include one or more optical elements such as focusing lenses 126 or one or more mirrors 128. In some embodiments, the optical elements 126, 128 can be individual elements in free space or can be contained within a single housing. In some embodiments, the optical elements can be integrally formed. In some embodiments, the reference arm 120 can also include an actuator to adjust the position of the focusing lenses 126, the mirror 128, or both to change a path length of the reference arm 120. In some embodiments, the actuator is a movable mirror. In embodiments that include an actuator, the actuator can be used to adjust the path length of the reference arm 120 to perform time-domain OCT. In such embodiments, the light source 110 can be held at a constant wavelength without scanning.

In use, light from the light source 110 for excitation of fluorescence in the portion 152 of the object 150 travels along the shared path 111 from the light source 110 to the optical circulator 112, travels along the shared path 121 from the optical circulator 112 to the beam splitter 125, travels along the shared path 124 from the beam splitter 125 to the WDM 130, and travels along the common optical path 132 including the optical fiber 134 to the portion 152 of the object 150.

Fluorescence is emitted from the object 150 and collected in the optical fiber 134 to return through the fluorescence measurement subsystem. The emitted light passes along the common optical path 132 to the WDM 130. As described above, the WDM directs the fluorescence along the path 142 to a filter 144. In accordance with some embodiments, the filter 144 can remove unwanted wavelengths from the fluorescence light. In some embodiments, the filter 144 is a cut-on (i.e., a long-pass) filter. In some embodiments, a cut-on frequency of the cut-on filter lies in the range from 825 nm to 925 nm. In some embodiments with multiple fluorescent contrast agents, one or more filters 144 in the system include one or more bandpass filters. In some embodiments the filter 144 is a notch blocking filter to block a band of wavelengths corresponding to the illumination light from the light source 110 while transmitting wavelengths corresponding to fluorescence contrast agents at wavelengths both greater than and less than the blocked band of wavelengths. In some embodiments the detector 162 is chosen to be preferentially sensitive to wavelengths corresponding to the fluorescence light and not wavelengths corresponding to illumination light from the light source 110. In these embodiments, the filter 144 is not required.

The portion of the fluorescence that passes through the filter 144 proceeds on a path 143 to the second detector 162. In some embodiments, the spectrometer 168 can be placed along the path 143 before the second detector 162 to spectrally separate or disperse the light. The second detector 162 detects the fluorescence and produces an output signal. In some embodiments, the second detector 162 can be an Indium Gallium Arsenide (InGaAs) photodiode. The signals output from the second detector 162 are received by the computing device 165 to produce fluorescence image data. In some embodiments, the first detector or the second detector can be a spectrally resolved detector array such as a charge-coupled device (CCD).

In some embodiments, the system 100 includes a cladding-mode stripper 136 or other optical extraction device to separate light that is primarily confined in the core of the optical fiber 134 from light that is primarily confined in a second layer or the outer cladding of the optical fiber 134. In embodiments that use a cladding-mode stripper 136, fluorescence can be directed on a path 145 directly to the filter 144 and on to detector 162 while the reflected light remains in the core of optical fiber 132 and is directed to the beamsplitter 125. In these embodiments, the WDM 130 and optical path 142 may be omitted and the optical fiber 132 may be connected to path 124.

In FIGS. 3A-3C, an embodiment of an imaging system 100′ is illustrated that includes a shared detector 160. In accordance with some embodiments, the optical coherence tomography subsystem 90′ uses the shared detector 160 to detect collected reflected light from the object 150 and reference light from the reference arm 120. The fluorescence measurement subsystem 95′ uses the shared detector 160 to detect fluorescence emitted from the object 150.

In some embodiments, the optical coherence tomography subsystem 90′ can include the optical circulator 112, the polarization controller 113, optical paths 111, 121, 122, 123, 132, 164, the k-trigger signal 166, the beam splitter 125, the reference arm 120 including lenses 126 and mirrors 128, the optical fiber 134, the shared detector 160, and the computing device 165 as shown in FIG. 3A.

In some embodiments, the fluorescence measurement subsystem 95′ can include the beam splitter 125, the optical circulator 112, the optical fiber 134, the detector 160, the polarization controller 113, optical paths 111, 121, 123, 132, 164, the k-trigger signal 166 and the computing device 165 as shown in FIG. 3B.

Prior to illuminating and exciting fluorescence in the object 150, the optical path in system 100′ is similar to that in system 100 of FIG. 2C as shown in FIG. 3C. Specifically, light travels on the path 111 from the light source 110 to the optical circulator 112 and then passes along the path 121 to the beam splitter 125. In some embodiments, a polarization controller 113 can be placed along the path 111 to allow adjustment of the polarization of the light from the light source 110. The polarization controller 113 can include manual adjustment or automated adjustment. In some embodiments, the beam splitter 125 is a 50/50 beam splitter. In some embodiments, the beam splitter 125 divides the light into unequal portions, e.g., a 75/25 beam splitter or a 90/10 beam splitter. A portion of the light exiting the beam splitter 125 follows path 122 to the reference arm 120 while the remaining portion of the light follows a path 132 to the optical fiber 134 to illuminate and excite fluorescence in the portion 152 of the object 150. Light returning from the reference arm 120 passes back along path 122 to the beam splitter 125.

Reflected collected light and emitted fluorescence return from the object 150 through the optical fiber 134 and along path 132 to the beam splitter 125. Upon reaching the beam splitter 125, the reference light, the collected reflected light, and the fluorescence are split by the beam splitter 125 directing a portion along onto a path 123 to the shared detector 160 and the remaining portion along the path 121 back to the optical circulator 112. The optical circulator 112 passes the remaining portion of reference light, the collected reflected light, and the fluorescence received from path 121 along a path 164 to the shared detector 160. The reference light and collected reflected light combine interferometrically at the shared detector 160 to form an OCT signal detected by the shared detector 160. Output from the shared detector 160 can be received by the computing device 165 to produce OCT data. The shared detector 160 can detect the fluorescence and generate a signal. Output signals from the detector 160 can be received by the computing device 165 to produce fluorescence image data. In some embodiments, the shared detector 160 can be an Indium Gallium Arsenide (InGaAs) photodiode.

In some embodiments, the shared detector 160 is a balanced photodetector such as a balanced photo-diode. The balanced photodetector can operate in two modes: difference mode and summation mode. In some embodiments, when the balanced photodetector is operated in difference mode, the output signal is representative of OCT data and, when the balanced photodetector is operated in summation mode, the output signal is representative of fluorescence image data. In some embodiments, the balanced photodetector can switch between difference and summation modes one or more times during data acquisition. In some embodiments, the balanced photodiode can output the difference mode signal and the summation mode signal synchronously. In some embodiments, the detector signals are temporally filtered before sum and difference operations are performed, providing separate sum and difference outputs for slowly varying and rapidly varying optical signals. In some embodiments slowly varying optical signals produce fluorescence data and rapidly varying signals produce OCT data.

Use of the common optical fiber 134 can provide co-registration of the collected reflected light and the collected emitted light at the shared detector 160. In particular, passage of the collected reflected light and collected emitted light pass through the same distal optics 138 (if present) and the same optical fiber 134 at the object 150 causes the magnification and position of the field of view for each of the two modes to be identical. Co-registration of the collected reflected light and the collected emitted light can improve computational speed during image reconstruction by the computing device 165 because additional processing steps including landmark identification or image shifting can be avoided or minimized.

As compared with system 100 of FIG. 2C, the system 100′ illustrated in FIG. 3C lacks some components such as the WDM 130, the filter 144, and the second detector 162. The removal of these components can provide a more streamlined optical setup with reduced maintenance. In addition, the greater overlap between the optical coherence tomography subsystem and the fluorescence measurement subsystem can reduce misalignment issues.

The fluorescence contrast agent 155 absorbs light produced by the light source 110 and, in turn, emits light that is detected by the detector 160 or second detector 162 in some embodiments. In some embodiments, the wavelength range for excitation of the fluorescence contrast agent 155 can include a wavelength of light generated by the light source 110. Although described extensively herein as a “fluorescence” contrast agent, the mechanism by which the fluorescence contrast agent 155 absorbs and emits light can include, and is not limited to, fluorescence, phosphorescence, plasmon resonance, or other suitable forms of luminescence, including via multiphoton excitation. In some embodiments, the system 100 includes the fluorescence contrast agent 155 targeted to a specific binding target on the object 150. In various embodiments, the fluorescence contrast agent 155 can be disposed on a surface of the object 150. In embodiments where the object 150 is a tissue, the fluorescence contrast agent 155 can be applied systemically to the host of the tissue or can be applied using other application techniques such as intraperitoneal lavage.

In accordance with various embodiments, the fluorescence contrast agent 155 can have any suitable composition that emits light including, but not limited to, single-walled carbon nanotubes, metallic nanoparticles, polymer nanoparticles, downconversion or upconversion nanoparticles such as lanthanide-doped fluorides like sodium yttrium fluoride (NaYF₄), quantum dots, fluorescent dyes, or phosphors. The fluorescence contrast agent 155 can include a photoluminescent nanostructure. In some embodiments, the fluorescence contrast agent 155 can emit at one or more emission wavelengths in the range of 1100 nm to 1800 nm.

In some embodiments, the system 100 can collect fluorescence light from one or more identified or unidentified endogenous constituents of the object 150. In embodiments where the object 150 is a tissue, endogenous constituents of the object 150 can include, but are not limited to, collagen, elastin, nicotinamide adenine dinucleotide phosphate (NADP or NADPH), or flavins. For example, many as-yet unidentified autofluorophores are present in the liver. In some embodiments, the light source 110 can operate under conditions sufficient to excite the fluorescence contrast agent 155 via two-photon excitation.

In some embodiments, the fluorescence contrast agent 155 can emit light within the near infrared II (NIR-II) wavelength range defined as the range from 1000 nm to 1400 nm. Single-walled carbon nanotubes (SWNTs) and downconversion/upconversion nanoparticles are examples of contrast agents that can emit in the NIR-II window. Some embodiments include fluorescence contrast agents 155 that emit in NIR-II, and that exhibit large Stokes shift between excitation and emission wavelengths, ultralow autofluorescence background, relative insensitivity to photobleaching compared to organic dyes, the ability to be functionalized with targeting and/or drug delivery agents, and high optical absorbance in the near infrared wavelength range (650-950 nm), which offers the possibility of photothermal therapy. In accordance with some embodiments, the attenuation range of the photoluminescent or fluorescence signal from the fluorescence contrast agent 155 is similar to the attenuation depth of the illumination light used for OCT imaging of the object 150. In some embodiments, this attenuation range is up to several millimeters. In some embodiments that use a single-mode optical fiber 134, the fluorescence light generated by the fluorescence contrast agent 155 can produces a sufficiently high enough signal-to-noise ratio to mitigate signal loss as the fluorescence light passes through the cladding of the optical fiber 134.

In accordance with various embodiments, the fluorescence contrast agent 155 can have a Stokes shift of between 100 nm and 1100 nm or, more preferably, between 100 nm and 600 nm. A very large Stokes shift to emission wavelengths in the NIR-II can prevent spectral leakage of fluorescence into the OCT imaging channel that can lead to ambiguities during OCT data reconstruction. A discussion of fluorescence contrast agents that emit in the NIR-II window including single-walled carbon nanotubes is found in U.S. patent application Ser. No. 13/755,613 filed on Jan. 31, 2013 and published as US 2013/0230464, the entire contents of which is incorporated herein by reference.

In some embodiments, the fluorescence contrast agents 155 can be bound to functionalized bacteriophages that bind to cancer-specific tumors. The tumor-type specificity of these functionalized probes, along with the unique fluorescence emission wavelengths for specific single-walled carbon nanotube enantiomers, can provide “tunable” fluorescence signatures with extremely large Stokes shifts from the excitation frequency. An example of the use of M13 phage-functionalized single-walled carbon nanotubes may be found in “M13 phage-functionalized single-walled carbon nanotubes as nanoprobes for second near-infrared window fluorescence imaging of targeted tumors” by H. Yi et al., Nano Lett. 2012 Mar. 14; 12(3): 1176-1183, the entire contents of which is incorporated herein by reference.

In some embodiments, the fluorescence image data generated by the computing device 165 can serve as markers for specific tumor types. For example, there are several subtypes of ovarian cancer. An individual chiral enantiomer or a subclass of wavelength-tunable fluorescence contrast agents 155 may thus be used to develop a functionalized probe specific to a single cancer subtype. Each cancer subtype would then fluoresce at a different wavelength in the NIR-II window. Importantly, each of the different fluorescence contrast agent enantiomers can be excited using the same single light source 110. In some embodiments, the fluorescence contrast agents 155 can be used not only to “tag” regions of OCT 3D imagery for further analysis but also to provide tumor specificity by identifying the specific disease present. Such a dual use can provide the capability for automated in-vivo patho-histology.

In some cases, very small tumors may not be identifiable in an OCT image via morphology alone. Because early detection of cancer tumors (i.e., detection when most of the tumors are still small) vastly improves patient outcomes, it is important to identify tumors at the earliest possible stage. In accordance with some embodiments of the present disclosure, dual-mode imaging using fluorescence contrast agents 155 can reliably identify tumors that are otherwise too small for an OCT subsystem to reliably detect.

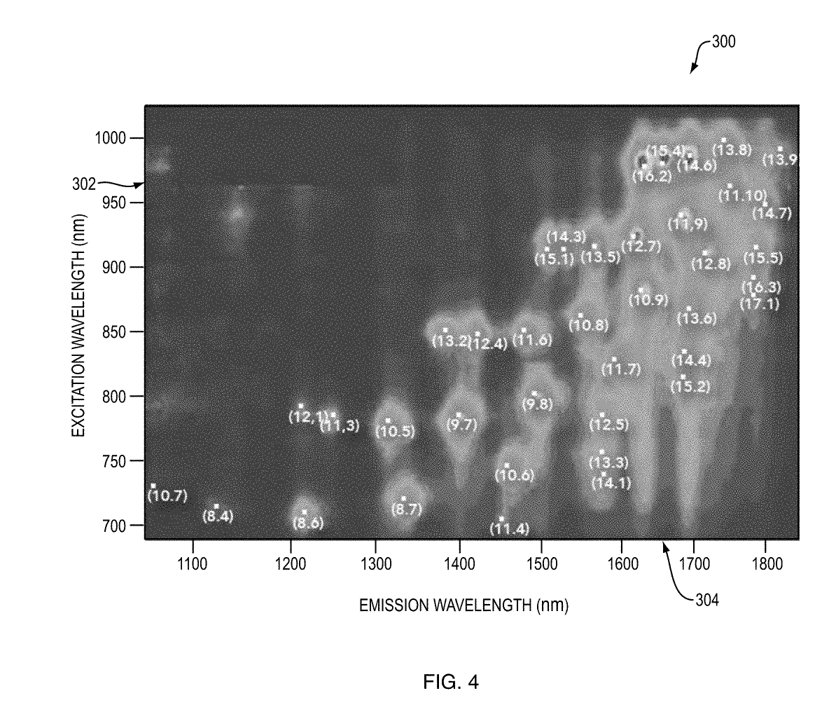

FIG. 4 illustrates an excitation-emission plot 300 for functionalized single-walled carbon nanotubes produced as described in US 2013/0230464 that can be used as fluorescence contrast agents 155 in accordance with some embodiments of the present disclosure.

The excitation wavelength is illustrated on the y-axis 302 while the emission wavelength is given on the x-axis 304. Each of the different “hot spots” on the plot 300 represents the optical properties of individual semiconducting single-walled carbon nanotube enantiomers (optical isomers), each with a different degree of angular skew. The degree of skew is represented by the chiral vector nomenclature (n.m) as shown in the plot and determines the bandgap for each enantiomer. The bandgap, in turn, dictates the fluorescent properties of that particular enantiomer. Carbon nanotubes with metallic or insulating properties are not fluorescent and do not appear on the plot. In accordance with various embodiments, the system 100 can include more than one fluorescence contrast agent 155 with differing excitation and or emission characteristics. As illustrated in FIG. 4, the functionalized probes exhibit Stokes shifts of 400 nm to 900 nm. The long-wavelength emission of these exemplary fluorescence contrast agents 155 can be well-separated spectrally from the light provided by the light source 110.

To avoid interference and reduce the need for filtering, the shift in wavelength of the fluorescence emitted from the object 150 as compared to the wavelength of the excitation light (e.g., the Stokes shift) should be large. In exemplary embodiments, the one or more emission wavelengths of the fluorescence contrast agent 155 are spectrally separated from one or more wavelengths of light provided by the light source 110 for illumination of the portion 152 of the object 150 and excitation of fluorescence. In some embodiments, the one or more emission wavelengths of the fluorescence contrast agent 155 are spectrally separated from the one or more wavelengths of light provided by the light source 110 for illumination of the portion of the object and excitation of fluorescence by a spectral separation falling in the range of 100 nm to 1100 nm. In some embodiments, the spectral separation falls in the range of 150 nm to 650 nm. By separating the wavelength ranges, spectral filtering requirements can be eased or even eliminated, and the collected reflected light and fluorescence may be more easily distinguished at the detector 160.

In accordance with various embodiments, the fluorescence contrast agent 155 can be functionalized to bind to a specific binding target on the object 150. In some embodiments, the fluorescence contrast agent 155 can include a targeting moiety such as proteins, antibodies, deoxyribonucleic acid (DNA), single- or double-stranded ribonucleic acid (RNA), carbohydrates, or any other suitable molecule.

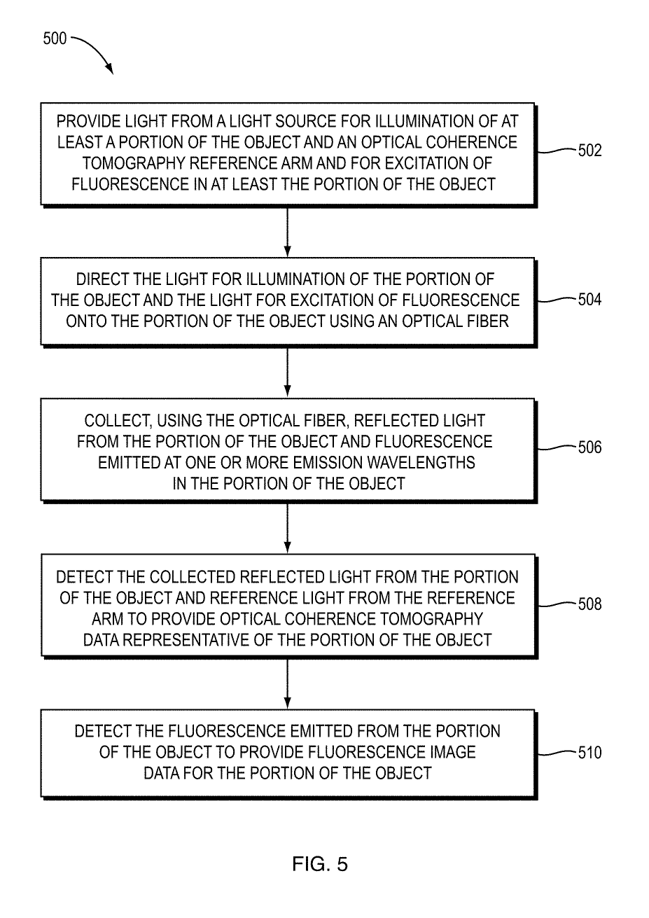

FIG. 5 illustrates a flowchart of a method 500 of imaging an object in accordance with various embodiments of the present disclosure. In step 502, light is provided from a light source for illumination of at least a portion of the object and an optical coherence tomography reference arm and for excitation of fluorescence in at least the portion of the object. The method 500 of imaging an object also includes directing the light for illumination of the portion of the object and the light for excitation of fluorescence onto the portion of the object using an optical fiber (step 504). Reflected light from the portion of the object and fluorescence emitted at one or more emission wavelengths in the portion of the object collected using the optical fiber in step 506. In some embodiments, the reflected light and the emitted fluorescence are collected synchronously. In step 508, the collected reflected light from the portion of the object and reference light from the reference arm are detected to provide OCT data representative of the portion of the object. In step 510, the fluorescence emitted from the portion of the object is detected to provide fluorescence image data for the portion of the object.

In some embodiments, the method 500 can also include disposing a fluorescence contrast agent that emits at the one or more emission wavelengths in the portion of the object. In some embodiments, the step of disposing the fluorescence contrast agent in the portion of the object can further include selectively binding a targeting moiety of the fluorescence contrast agent to a binding target in the portion of the object. In some embodiments, the fluorescence contrast agent can have a Stokes shift of between 100 nm and 1100 nm or between 100 nm and 600 nm. In accordance with some embodiments, the fluorescence contrast agent can be one or more of a downconversion nanoparticle, an upconversion nanoparticle, a single-walled carbon nanotube, or a photoluminescent nano structure.

Merely for illustrative purposes, the method 500 is described in greater detail below with reference to the systems 100 and 100′ appearing respectively in FIGS. 3 and 4. One skilled in the art, in view of the present disclosure, will appreciate that method 500 can be performed with other systems having more, fewer, or different components than those shown in FIGS. 3 and 4. In step 502, light source 110 provides light for illumination of at least the portion 152 of the object 150 and the optical coherence tomography reference arm 120 and for excitation of fluorescence in at least the portion 152 of the object 150. In some embodiments, the light source is a swept source and providing light in accordance with the method includes sweeping a wavelength of the light source. In some embodiments, the range of the wavelength sweep falls in a range of 750 nm to 850 nm. In step 504, the light for illumination of the portion 152 of the object 150 and the light for excitation of fluorescence are directed onto the portion 152 of the object 150 using an optical fiber 134.

Reflected light from the portion 152 of the object 150 and fluorescence emitted at one or more emission wavelengths in the portion 152 of the object 150 are synchronously collected using the optical fiber 134 in step 506. In some embodiments, the one or more emission wavelengths lie in a range from 1100 nm to 1800 nm. In some embodiments, the one or more emission wavelengths are spectrally separated from one or more wavelengths of the light provided by the light source 110 for illumination of the portion 152 of the object 150 and excitation of the fluorescence.

In some embodiments, at least a portion of the optical fiber 134 can be double-clad fiber as described above with reference to FIGS. 3 and 4. In such embodiments, the method 500 can include transmitting the collected reflected light in a single-mode inner core of the double-clad fiber and transmitting the fluorescence in a multi-mode outer core of the double-clad fiber. In some embodiments, at least a portion of the optical fiber 134 can be single-mode fiber as described above with reference to FIGS. 3 and 4. In such embodiments, the method 500 can include transmitting the collected reflected light in a single-mode inner core of the single-mode fiber and transmitting the fluorescence in a cladding of the single-mode fiber. In accordance with some embodiments, the optical fiber 134 can collect reflected light having a wavelength in the range of 750 nm to 850 nm and can collect fluorescence having a wavelength in the range of 1100 to 1800 nm.

The collected reflected light from the portion 152 of the object 150 and reference light from the reference arm 120 are detected to provide OCT data representative of the portion 152 of the object 150 in step 508. In some embodiments, the method 500 can include coupling the fluorescence out of the single-mode fiber using a cladding mode stripper 136 or other optical extraction device. In step 510, the fluorescence emitted from the portion 152 of the object 150 is detected to provide fluorescence image data for the portion 152 of the object 150. In accordance with some embodiments, the collected reflected light, the reference light, and the fluorescence are detected with the same detector 160. In other embodiments, the collected reflected light and the reference light are detected by the first detector 161 while the fluorescence is detected by the second detector 162.

In some embodiments, the method 500 produces spatially registered images that include the OCT data and the fluorescence image data. In some embodiments, a frequency sweep of the light source is synchronized to the signals acquired from the first detector or the shared detector using a k-trigger.

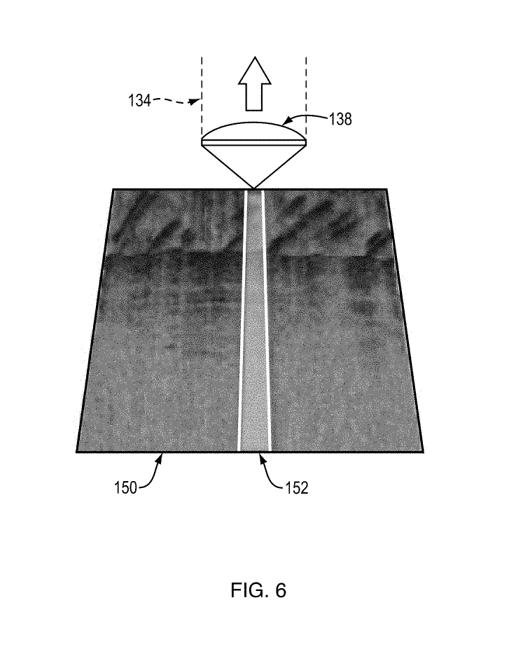

FIG. 6 schematically depicts an axial cross-section of a region of tissue, with the axial (depth) axis shown vertically, as the tissue is being imaged crosswise (left-to-right) by the system 100 presented herein in accordance with some embodiments. As described previously, the system 100 can include the optical fiber 134 to provide light and collect light at the object 150, in this case a tissue. In some embodiments, the system 100 can include distal optics 138 such as lenses and mirrors to direct the light out of the optical fiber 134 onto the object 150 and to collect light from the object 150 into the optical fiber 134. In some embodiments, the distal optics 138 can be moveable such as through a micromotor or piezo stack to scan the direction at which the light emerges from the optical fiber 134. For example, the portion 152 of the object 150 that is shown as illuminated in FIG. 6 can correspond to the lateral region of OCT axial scans in which fluorescence is detected. In accordance with some embodiments, the system 100 can scan the light beam in one dimension or two dimensions to form line scans, raster scans, or any other suitable scanning architecture.

In accordance with some embodiments, the system 100 can acquire OCT data at spatial resolutions as low as 2 μm in the lateral direction and 14 μm in the axial direction. The fluorescence image data can also be acquired at a spatial resolution of 2 μm in the lateral direction and 14 μm in the axial direction. However, the diffuse nature of the fluorescence signal may lead to low signal counts in each pixel at such low resolution. In some embodiments, the fluorescence image data can be binned or integrated to produce data with a spatial resolution of about 100-500 μm.

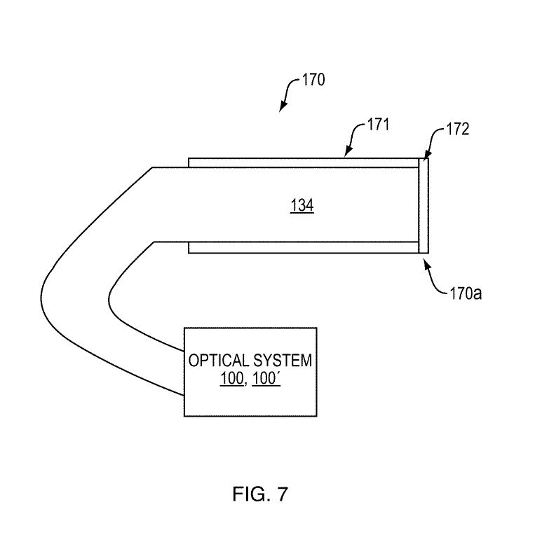

FIG. 7 depicts the endoscope 170 for use with the imaging system 100, 100′ in some embodiments. The endoscope 170 can include a sheath 171 and a distal window 172. In some embodiments, the distal window can have a hemispherical shape. In some embodiments, the sheath 171 can be transparent. In some embodiments, the optical fiber 134 extends into the sheath 171 to collect reflected and emitted light from the object. In some embodiments, a detachable portion of the optical fiber 134 is embedded within the sheath 171 so that the endoscope 170 may be sterilizable or disposable. The distal window 172 can seal a distal end 170a of the endoscope 170 to prevent, for example, moisture infiltration in some embodiments. The distal window 172 may also be shaped in some embodiments as a lens or other beamshaping element to help collect the reflected and emitted light from the object 150 into the optical fiber 134. In some embodiments, the image is collected through the transparent sheath 171 instead of through the distal window 172. In some embodiments, the transparent sheath and distal window are formed as one transparent component, and the image is collected either through the distal window or laterally through the transparent sheath. In some embodiments, a diameter of the distal end 170a of the endoscope 170 can be small enough to fit within lumens such as small blood vessels or fallopian tubes.

In some embodiments, the OCT subsystem is a Fourier-domain OCT subsystem. In such embodiments, the light source may be a low coherence polychromatic light source. In the OCT subsystem, the detector can be a spectrally resolved detector array such as a charge-coupled device (CCD). The OCT subsystem can include a diffraction grating prior to a detector.

FIG. 8 is a block diagram of the computing device 165 that may be used to implement some embodiments of the methods and systems described herein. For example, in some embodiments, computing device 165 is used for image reconstruction based on the OCT data and the fluorescence image data. Descriptions and elements of the computing device 165 below may be applicable to any computing device described above with reference to previous embodiments. The computing device 165 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives, one or more solid state disks), and the like. For example, memory 806 included in the computing device 165 may store computer-readable and computer-executable instructions or software for implementing exemplary embodiments of the imaging system 100. The computing device 165 also includes a configurable or programmable processor 868 and associated core(s) 804 and may include one or more additional configurable or programmable processor(s) 802′ and associated core(s) 804′ (for example, in the case of computer systems having multiple processors or cores), for executing computer-readable and computer-executable instructions or software stored in the memory 806 and other programs for controlling system hardware. Processor 868 and processor(s) 802′ may each be a single core processor or multiple core (804 and 804′) processor.

Virtualization may be employed in the computing device 165 so that infrastructure and resources in the computing device may be shared dynamically. A virtual machine 814 may be provided to handle a process running on multiple processors so that the process appears to be using only one computing resource rather than multiple computing resources. Multiple virtual machines may also be used with one processor.

Memory 806 may include a computer system memory or random access memory, such as DRAM, SRAM, EDO RAM, and the like. Memory 806 may include other types of memory as well, or combinations thereof. In some embodiments, the memory 806 can be used to store OCT data 805 or fluorescence image data 807.

A user may interact with the computing device 165 through the visual display device 867 such as a computer monitor, which may display one or more graphical user interfaces 822, that may be provided in accordance with exemplary embodiments. The computing device 165 may include other I/O devices for receiving input from a user, for example, a keyboard or any suitable multi-point touch interface 808, a pointing device 810 (e.g., a mouse), a microphone 828, or an image capturing device 832 (e.g., a camera or scanner). The multi-point touch interface 808 (e.g., keyboard, pin pad, scanner, touch-screen, etc.) and the pointing device 810 (e.g., mouse, stylus pen, etc.) may be coupled to the visual display device 867. The computing device 165 may include other suitable conventional I/O peripherals. In some embodiments, the computing device 165 can include an I/O device 835 to receive signals or image data from the shared detector 160, the first detector 161, or the second detector 162. In some embodiments, the I/O device 835 consists of a series of foot-actuated controls that can be accessed when the user's hands are otherwise occupied (during surgery, for example).

The computing device 165 may also include one or more storage devices 824, such as a hard-drive, CD-ROM, or other computer readable media, for storing data and computer-readable instructions or software that implement exemplary embodiments of an imaging system 100. For example, the storage 824 can store one or more implementations of automated processing algorithm codes 823 to generate images containing OCT data and fluorescence image data. Exemplary storage device 824 may also store one or more databases for storing any suitable information required to implement exemplary embodiments. For example, exemplary storage device 824 can store one or more databases 826 for storing information, such as object identification information or metadata, probe parameters, k-trigger timing, patient data, or any other information to be used by embodiments of the system 100. The databases may be updated manually or automatically at any suitable time to add, delete, or update one or more data items in the databases.

The computing device 165 can include a network interface 812 that can be used to transmit or receive data, or communicate with other devices, in any of the example embodiments described herein. Network interface 812 can be configured to interface via one or more network devices 820 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. In exemplary embodiments, the computing device 165 can include one or more antennas 830 to facilitate wireless communication (e.g., via the network interface) between the computing device 165 and a network. The network interface 812 may include a built-in network adapter, network interface card, PCMCIA network card, card bus network adapter, wireless network adapter, USB network adapter, modem or any other device suitable for interfacing the computing device 165 to any type of network capable of communication and performing the operations described herein. Moreover, the computing device 165 may be any computer system, such as a workstation, desktop computer, server, laptop, handheld computer, tablet computer (e.g., the IPAD™ tablet computer), mobile computing or communication device (e.g., the IPHONE™ communication device), internal corporate devices, or other form of computing or telecommunications device that is capable of communication and that has sufficient processor power and memory capacity to perform the operations described herein.

The computing device 165 may run any operating system 816, such as any of the versions of the MICROSOFT® WINDOWS® operating systems, the different releases of the Unix and Linux operating systems, any version of the MACOS® for Macintosh computers, any embedded operating system, any real-time operating system, any open source operating system, any proprietary operating system, or any other operating system capable of running on the computing device and performing the operations described herein. In exemplary embodiments, the operating system 816 may be run in native mode or emulated mode. In an exemplary embodiment, the operating system 816 may be run on one or more cloud machine instances.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements, device components or method steps, those elements, components or steps may be replaced with a single element, component or step. Likewise, a single element, component or step may be replaced with a plurality of elements, components or steps that serve the same purpose. Moreover, while exemplary embodiments have been shown and described with references to particular embodiments thereof, those of ordinary skill in the art will understand that various substitutions and alterations in form and detail may be made therein without departing from the scope of the invention. Further still, other aspects, functions and advantages are also within the scope of the invention.

An exemplary flowchart is provided herein for illustrative purposes and is a non-limiting example of a method. One of ordinary skill in the art will recognize that exemplary methods may include more or fewer steps than those illustrated in the exemplary flowcharts, and that the steps in the exemplary flowcharts may be performed in a different order than the order shown in the illustrative flowcharts.

1. An imaging system for generating fluorescence image data and optical coherence tomography data of an object, the imaging system comprising:
a light source configured to provide light for illumination of at least a portion of an object and for excitation of fluorescence in at least the portion of the object;
an optical coherence tomography subsystem configured to provide optical coherence tomography data of the portion of the object and configured to use light from the light source; and
a fluorescence measurement subsystem configured to provide fluorescence image data of the portion of the object and configured to use light from the light source.

2. The imaging system of claim 1, wherein the optical coherence tomography subsystem and the fluorescence imaging subsystem are configured to synchronously provide corresponding OCT data and fluorescence image data.

3. The imaging system of claim 1, wherein the optical coherence tomography subsystem and the fluorescence imaging subsystem synchronously receive reflected light from the portion of the object and fluorescence light emitted from the portion of the object, respectively.

4. The imaging system of claim 1, further comprising an optical fiber configured to:
direct the illumination and excitation light onto the portion of the object; and
collect reflected light from the portion of the object and fluorescence emitted at one or more emission wavelengths from the portion of the object.

5. The imaging system of claim 4, wherein at least a portion of the optical fiber is disposed within an endoscope.

6. The imaging system of claim 4, wherein the optical coherence tomography subsystem comprises a reference arm that receives light from the light source.

7. The imaging system of claim 6, further comprising a detector, wherein the imaging system is configured such that the detector receives the collected reflected light from the portion of the object and reference light from the reference arm for providing optical coherence tomography data representative of the portion of the object and receives fluorescence emitted from the portion of the object for providing fluorescence image data from the portion of the object.

8. The imaging system of claim 7, wherein the OCT data and fluorescence data are separated using temporal filtering.

9. The imaging system of claim 7, wherein the detector is a balanced photo-diode that provides optical coherence tomography data when operated in a difference mode and provides fluorescence image data when operated in a summation mode.

10. The imaging system of claim 6, wherein the optical coherence tomography subsystem further comprises a first detector, wherein the optical coherence tomography subsystem is configured such that the first detector receives the collected reflected light from the portion of the object and reference light from the reference arm to provide optical coherence tomography data representative of the portion of the object.

11. The imaging system of claim 10, wherein the first detector is a balanced photo-diode.

12. The imaging system of claim 10, wherein the fluorescence measurement subsystem further comprises a second detector, wherein the fluorescence measurement subsystem is configured such that the second detector receives fluorescence emitted from the portion of the object to provide fluorescence image data from the portion of the object.

13. The imaging system of claim 6, wherein the optical coherence tomography subsystem further comprises an actuator configured to change an optical path length in the reference arm.

14. The imaging system of claim 4, wherein at least a portion of the optical fiber is double-clad fiber.

15. The imaging system of claim 14, wherein the optical fiber is configured to collect reflected light having a wavelength in the range of 750 nm to 850 nm and configured to collect fluorescence having a wavelength in the range of 1100 nm to 1800 nm.

16. The imaging system of claim 14, wherein the imaging system is configured such that the collected reflected light travels in a single-mode inner core of the double-clad fiber and the fluorescence light travels in a multi-mode outer core of the double-clad fiber.

17. The imaging system of claim 4, wherein at least a portion of the optical fiber is single-mode fiber.

18. The imaging system of claim 17, wherein the optical fiber is configured to collect reflected light having a wavelength in the range of 750 nm to 850 nm and configured to collect fluorescence light having a wavelength in the range of 1100 nm to 1800 nm.

19. The imaging system of claim 17, wherein the imaging system is configured such that the collected reflected light travels in a single-mode inner core of the single-mode fiber and the fluorescence light travels in a cladding of the single-mode fiber.

20. The imaging system of claim 19, further comprising a cladding mode stripper or other optical extraction device configured to couple fluorescence light out of the single-mode fiber.

21. The imaging system of claim 4, further comprising a fluorescence contrast agent in the portion of the object, wherein one or more emission wavelengths of the fluorescence contrast agent are spectrally separated from one or more wavelengths of light provided by the light source for illumination of the portion of the object and excitation of fluorescence.

22. The imaging system of claim 21, wherein the one or more emission wavelengths of the fluorescence contrast agent are spectrally separated from the one or more wavelengths of light provided by the light source for illumination of the portion of the object and excitation of fluorescence by a spectral separation falling in a range of 100 nm to 1100 nm.

23. The imaging system of claim 22, wherein the spectral separation falls in a range of 150 nm to 650 nm.

24. The imaging system of claim 1, further comprising a fluorescence contrast agent in the portion of the object, wherein the fluorescence contrast agent has a Stokes shift falling in a range of 100 nm to 1100 nm.

25. The imaging system of claim 24, wherein the fluorescence contrast agent has a Stokes shift falling in a range of 100 nm to 600 nm.

26. The imaging system of claim 1, further comprising a fluorescence contrast agent in the portion of the object, wherein the fluorescence contrast agent is a single-walled carbon nanotube.

27. The imaging system of claim 1, further comprising a fluorescence contrast agent in the portion of the object, wherein the fluorescence contrast agent is a downconversion nanoparticle or an upconversion nanoparticle.

28. The imaging system of claim 1, further comprising a fluorescence contrast agent in the portion of the object, wherein the fluorescence contrast agent is a photoluminescent nanostructure.

29. The imaging system of claim 1, further comprising a fluorescence contrast agent in the portion of the object, wherein one or more emission wavelengths of the fluorescence contrast agent lie in the range of 1100 nm to 1800 nm.

30. The imaging system of claim 1, wherein the light source is a frequency-swept source.

31. The imaging system of claim 30, wherein wavelengths of light provided by the frequency-swept light source for illumination of at least a portion of the object and excitation of fluorescence in at least the portion of the object lie in a range of 750 nm to 850 nm.

32. The imaging system of claim 1, further comprising a computer to receive the optical coherence tomography data and the fluorescence image data, the computer configured to produce spatially registered images including the optical coherence tomography data and the fluorescence image data.

33. A method of imaging an object, comprising:
providing light from a light source for illumination of at least a portion of the object and an optical coherence tomography reference arm and for excitation of fluorescence in at least the portion of the object;
directing the light for illumination of the portion of the object and the light for excitation of fluorescence onto the portion of the object using an optical fiber;
collecting, using the optical fiber, reflected light from the portion of the object and fluorescence emitted at one or more emission wavelengths in the portion of the object;
detecting the collected reflected light from the portion of the object and reference light from the reference arm to provide optical coherence tomography data representative of the portion of the object; and
detecting the fluorescence emitted from the portion of the object to provide fluorescence image data for the portion of the object.

34. The method of claim 33, wherein the optical coherence tomography data and the fluorescence image data are provided synchronously.

35. The method of claim 33, wherein the reflected light from the portion of the object and fluorescence emitted in the portion of the object are collected synchronously.

36. The method of claim 33, wherein at least a portion of the optical fiber is disposed within an endoscope.

37. The method of claim 33, wherein the collected reflected light and the reference light are detected by a first detector and the fluorescence is detected by a second detector.

38. The method of claim 33, wherein the collected reflected light, the reference light, and the fluorescence are detected by a same detector.

39. The method of claim 38, wherein the OCT data and fluorescence data are separated using temporal filtering.

40. The method of claim 38, wherein the detector is a balanced photo-diode.

41. The method of claim 40, wherein the OCT data and fluorescence data are separated using temporal filtering.

42. The method of claim 40, wherein detecting the collected reflected light from the portion of the object and the reference light from the reference arm includes operating the balanced photo-diode in a difference mode; and
wherein detecting the fluorescence emitted from the portion of the object includes operating the balanced photo-diode in a summation mode.

43. The method of claim 33, further comprising disposing a fluorescence contrast agent that emits at the one or more emission wavelengths in the portion of the object.

44. The method of claim 43, wherein disposing a fluorescence contrast agent in the portion of the object comprises selectively binding a targeting moiety of the fluorescence contrast agent to a binding target in the portion of the object.

45. The method of claim 43, wherein the fluorescence contrast agent has a Stokes shift of between 100 nm and 1100 nm.

46. The method of claim 45, wherein the fluorescence contrast agent has a Stokes shift of between 100 nm and 600 nm.

47. The method of claim 43, wherein the fluorescence contrast agent is one or more of: a downconversion nanoparticle or an upconversion nanoparticle.

48. The method of claim 43, wherein the fluorescence contrast agent is a single-walled carbon nanotube.

49. The method of claim 43, wherein the fluorescence contrast agent is a photoluminescent nanostructure.

50. The method of claim 33, wherein the one or more emission wavelengths lie in the range from 1100 nm to 1800 nm.

51. The method of claim 33, wherein the one or more emission wavelengths are spectrally separated from one or more wavelengths of the light provided by the light source for illumination of the portion of the object and excitation of the fluorescence.

52. The method of claim 33, wherein providing light from a light source for illumination of at least a portion of the object and a reference arm of an imaging system and for excitation of fluorescence in the portion of the object comprises sweeping a wavelength of the light source.

53. The method of claim 52, wherein the range of the wavelength sweep is included within the range of 750 nm and 850 nm.

54. The method of claim 33, wherein at least a portion of the optical fiber is double-clad fiber; and
wherein the method further comprises transmitting the collected reflected light in a single-mode inner core of the double-clad fiber and transmitting the fluorescence in a multi-mode outer core of the double-clad fiber.

55. The method of claim 54, wherein the optical fiber collects reflected light having a wavelength in the range of 750 nm to 850 nm and collects fluorescence having a wavelength in the range of 1100 nm to 1800 nm.

56. The method of claim 33, wherein at least a portion of the optical fiber is single-mode fiber; and
wherein the method further comprises transmitting the collected reflected light in a single-mode inner core of the single-mode fiber and transmitting the fluorescence in a cladding of the single-mode fiber.

57. The method of claim 56, wherein the optical fiber collects reflected light having a wavelength in the range of 750 nm to 850 nm and collects fluorescence having a wavelength in the range of 1100 nm to 1800 nm.

58. The method of claim 56, further comprising coupling the fluorescence out of the single-mode fiber using a cladding mode stripper or other optical extraction device.

59. The method of claim 33, further comprising producing spatially registered images that include the optical coherence tomography data and the fluorescence image data.
